(19) United States
(12) Patent Application Publication
(10) Pub. No.: US 2011/0288444 A1
(43) Pub. Date: Nov. 24, 2011

(54) Eschar Prevention Device

(76) Inventors: Yohan Payan, Allevard (FR); Jacques Demongeot, Sassenage (FR); Jose-Octavio Vazquez-Buenosaires, Tuxtla Gutierrez (MX)

(21) Appl. No.: 11/630,417

(22) PCT Filed: Jun. 24, 2005
(86) PCT No.: PCT/FR2005/050490

§ 371 Date: Feb. 18, 2011

(30) Foreign Application Priority Data
Jun. 24, 2004 (FR) 0451319

(51) Int. Cl. A61B 5/00

(52) U.S. Cl. 600/587

(57) ABSTRACT

The invention relates to a device for preventing the formation of eschars in a support area, i.e the area on which a person in a seated or lying position rests. The invention comprises: pressure sensors which are disposed under the aforementioned support area; means for comparing each measurement supplied by any of the sensors to at least one threshold value; storage means for storing all of the measurements supplied by all of the sensors during a first period of time from the point at which the threshold value is exceeded by at least one measurement; means for processing the stored measurements, which, once the first time period has elapsed are used to determine movement instructions for the person in the seated or lying position such as to prevent the formation of eschars; a lingual electro-stimulation device for transmitting the aforementioned movement instructions to the person; and actuators which can be actuated by the person in the seated or lying position and which regulate the movement of said person

### FIELD OF THE INVENTION

The present invention relates to equipments of wheel-chairs for physically disabled, para- or tetraplegic persons, as well as beds for disabled or ill persons. More specifically, the present invention relates to a device for preventing eschars.

### DISCUSSION OF PRIOR ART

In seated or lying position, a non-disabled individual never stops being moved by reflex movements, most often imperceptible, to move this individual's bearing areas, which enables avoiding the forming of eschars. However, a medullar wounded or para- or tetraplegic physically disabled person, partially or totally immobilized in seated or lying position, does not have such reflex motions.

For spinal cord injured or para- or tetraplegic physically disabled persons, a clinical training device has been provided. This device is used in a hospital environment for a person who already has had eschars. The person is placed on a seating formed of a seat of a 43×43-cm dimension having its surface equipped with a matrix of 30×30 pressure sensors. The pressure sensors are regularly distributed across the seat surface. The sensors then provide a pressure level color map. This map is then observed in real time by the patient and by a specialist who explains to the seated person how to modify his or her position to take an ideal position. In such an ideal position, which essentially depends on the person's morphology and/on handicap, the pressures axe substantially equally distributed. Eschar risks are then considerably decreased, or even null.

Such a device has many disadvantages, first it is most of the time implemented only after eschars have appeared while the resorption of this type of necrosis is particularly long and painful for a person who cannot stand up. Then, it requires for the seated person to go to a hospital center, which is sometimes not easily compatible with professional activities. It has further been observed that even though patients watch their position in their usual wheelchair in the form a few hours to a few days following their visit to the center, they very quickly stop watching their position and return to their usual bad positions resulting in the forming of new eschars. Further, the efficiency of such a periodic training seems to be inversely proportional to the patient's age.

The present invention accordingly aims at providing a device for preventing eschars which is simpler to implement.

The present invention aims at providing such a device which is embarked in the wheelchair of a physically disabled person.

The present invention also aims at providing such a device which adapts to a given patient's morphology.

### SUMMARY OF THE INVENTION

To achieve these objects, the present invention provides a device for preventing the forming of eschars at the level of a seated or lying person's bearing area, comprising:

pressure sensors under the bearing area;

means for comparing each measurement provided by any one of the sensors with at least one threshold value;

memorization means capable of memorizing all the measurements provided by all the sensors for a first time period from an exceeding of the threshold value by at least one measurement;

means for processing the memorized measurements capable of determining, once the first time period has elapsed, displacement instructions for the seated or lying person to avoid the forming of eschars;

a tongue display unit capable of transmitting the displacement instruction to the seated or lying person; and

actuators operable by the seated or lying person and capable of causing his or her displacement.

According to an embodiment of the present invention, the device further comprises:

means for determining after an exceeding of the threshold value for a given sensor whether the measurements subsequently provided by the given sensor become lower than the threshold value;

means for measuring the time for which the measurements provided by the sensor remain lower than the threshold value; and

means for interrupting for the given sensor any processing of memorization of the measurements and/or of transmission of instructions for this sensor if and as soon as the time reaches a second predetermined time period.

According to an embodiment of the present invention, the value of the first time period depends on the distance from the threshold value of the measurement having exceeded the threshold value.

According to an embodiment of the present invention, the threshold value is equal to 13.332.10³ Pa (100 mm Hg) and the first time period is equal to thirty minutes if the measured value is lower than 15.332.10³ Pa (115 mm Hg), the first time period is equal to fifteen minutes if the measured value ranges between 15.332.10³ Pa (115 mm Hg) and 17.331.10³ Pa (130 mm Hg), and the first time period is equal to ten minutes if the measured value is greater than 17.331.10³ Pa (130 mm Hg).

According to an embodiment of the present invention, the pressure sensors are distributed under the bearing area at the intersection of the lines and columns of a matrix with twelve lines and twelve columns, divided into two sub-matrixes with twelve lines and six columns, the interval between two successive lines of each sub-matrix being, from the rear to the front of the bearing area, 2 cm, 1.5 cm, 1.5 cm, 1 cm, 1 cm, 1 cm, 1.5 cm, 1.5 cm, 2 cm, 3 cm, and 4 cm, and the interval between two successive columns of each sub-matrix being 3.5 cm, 3 cm, 2 cm, 3 cm, and 3.5 cm.

According to an embodiment of the present invention, the two sub-matrixes are adjacent.

According to an embodiment of the present invention, the two sub-matrixes are separate.

According to an embodiment of the present invention, the interval between the two sub-matrixes is settable on the bearing area according to the seated or lying person's morphology.

According to an embodiment of the present invention, the tongue display unit is formed of a square matrix of thirty-six electrodes.

According to an embodiment of the present invention, the means for processing the measurements are capable of calculating averages of the measurements memorized for the first time period, of determining and of ordering among the obtained averages the maximum values exceeding the threshold value and of determining instructions for displacing the seated or lying person to suppress the maximum values.

### BRIEF DESCRIPTION OF THE DRAWINGS

The foregoing and other objects, features, and advantages of the present invention will be discussed in detail in the following non-limiting description of specific embodiments in connection with the accompanying drawings, among which:

FIG. 1 schematically illustrates eschar-forming risk areas;

FIG. 2 illustrates a sensor matrix used in a device according to the present invention;

FIG. 3 illustrates a first position-correction coding according to the present invention; and

FIG. 4 illustrates another position-correction coding according to the present invention.

### DETAILED DESCRIPTION

For clarity, same elements have been designated with same reference numerals in the different drawings. Further, the drawings are not to scale.

The present invention will be described hereafter in the case of a seated person. However, it should be understood by those skilled in the art that the device according to the present invention may also be used for a person confined to bed.

The present invention takes advantage of the inventors' observations on the existence of particularly sensitive areas for the forming of eschars at the level of a seated persona bearing area.

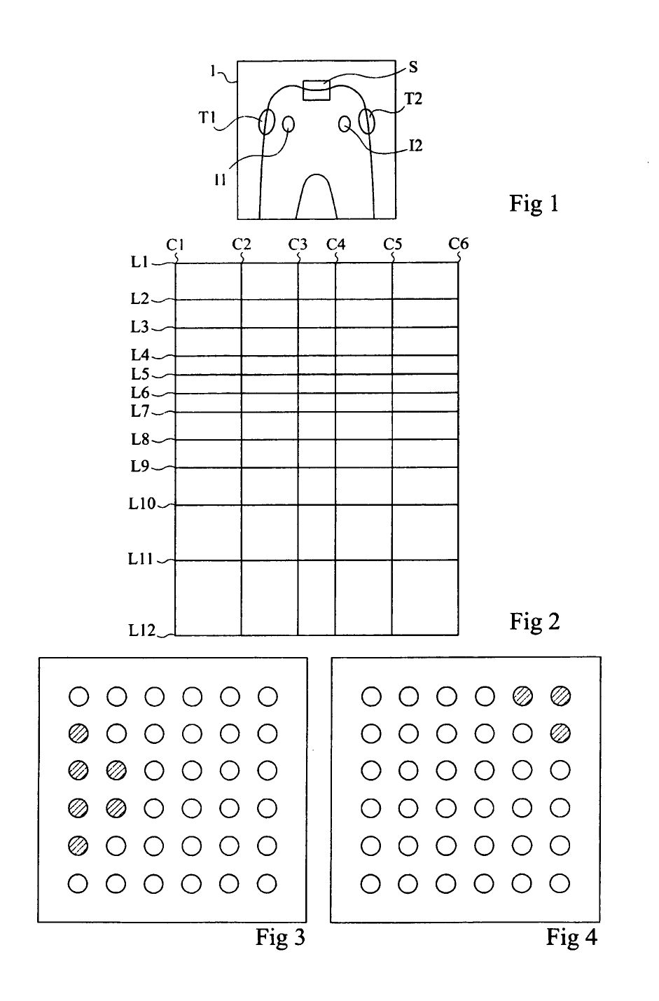

FIG. 1 schematically illustrates a seat 1 of a wheel-chair on which a person is seated. This person has five sensitive areas symmetrically distributed in three areas. These areas are bearing areas I1 and I2 of the left-hand and right-hand ischia, bearing areas T1 and T2 of the left-hand and right-hand trochanters, and central bearing area S of the sacrum.

The device according to the present invention comprises a matrix of 12×12 sensors formed of two sub-matrixes with twelve lines and six columns. The sensor matrix is placed on the seat of a wheelchair in the rear portion of the seating area of a standard 43×43-cm dimension and has a total 30×30-cm dimension. Each sub-matrix exhibits a same irregular distribution of the sensors selected to concentrate a larger number of sensors in the sensitive areas.

FIG. 2 schematically shows such a sub-matrix. From back to front, the twelve lines are numbered from L1 to L12. The columns are designated from left to right as C1, C2, C3, C4, C5, and C6. The interval separating two successive lines varies as follows:

L1-L2: 2 cm;

L2-L3: 1.5 cm;

L3-L4: 1.5 cm;

L4-L5: 1 cm;

L5-L6: 1 cm;

L6-L7: 1 cm;

L7-L8: 1.5 cm;

L8-L9: 1.5 cm;

L9-L10: 2 cm;

L10-L11: 3 cm;

L11-L12: 4 cm.

The interval between two successive columns is the following:

C1-C2: 3.5 cm;

C2-C3: 3 cm;

C3-C4: 2 cm;

C4-C5: 3 cm;

C5-C6: 3.5 cm.

According to an embodiment of the present invention, the two sub-matrixes are attached.

According to an embodiment of the present invention, the two sub-matrixes axe separate, provided with removable devices for attachment to the seat. This enables adapting to the seated person's morphology by modifying the interval between sub-matrixes so that they are each centered on an ischium.

The 12×12 sensors Sᵢⱼ distributed at the intersection of lines Lᵢ and columns Cⱼ provide pressure measurements Mᵢⱼ at the level of the seated person's bearing area. Measurements Mᵢⱼ are performed every second, that is, with a 1-Hz frequency.

The device according to the present invention comprises means for receiving measurements Mᵢⱼ and for comparing them with at least one threshold value. For example, measurements Mᵢⱼ are compared with three successive increasing threshold values. For example, the threshold values are pressures of 13.332.10³ Pa, 15.332.10³ Pa, and 17.331.10³ Pa (100, 115, and 130 millimeters of mercury).

The device according to the present invention comprises memorization means. Such memorization means are activated as soon as a measurement Mᵢⱼ exceeds one of the threshold values, and then memorize each group of measurements Mᵢⱼ provided by all the sensors Sᵢⱼ. Inside of each group (matrix) of measurements Mᵢⱼ, each measurement M_{id} takes up a location corresponding to the location of the sensor Sᵢⱼ which has provided it.

The groups of 144 instantaneous measurements Mᵢⱼ are memorized for a time period which depends on the distance of the measurement having caused the memorization from the threshold value. In the considered case where several threshold values are set, the memorization time depends on the initially-exceeded threshold value. The higher the threshold value, the shorter the memorization time. For example, when the measurement Mᵢⱼ having triggered the memorization is greater than 17.331.10³ Pa (130 mm Hg), the memorization time is ten minutes. When measurement Mᵢⱼ ranges between 15.332.10³ and 17.331.10³ Pa (115 and 130 mm Hg), the memorization time is 15 minutes. When measurement Mᵢⱼ ranges between 13.332.10³ and 15.332.10³ Pa (100 and 115 mm Hg), the time period is 30 minutes.

The device according to the present invention comprises means capable of selecting the memorization time period based on the result of the comparison and means capable of determining the elapsing of this time period.

The device according to the present invention also comprises means capable of processing, once the memorization time period has elapsed, all the measurement groups memorized during this time. More specifically, such processing means comprise means capable of calculating, for each sensor Sᵢⱼ, the average pressure value for the memorization time period. The average values are memorized in a group (matrix) of same dimension as instantaneous measurement groups Mᵢⱼ. The processing means further comprise comparison means for determining among all the average values the existence of maximum values greater than the lowest threshold value of 13.332.10³ Pa (100 mm Hg). The processing means further comprise means capable of ordering the different maximum values by increasing order.

The device according to the present invention comprises a means of truth table type capable of associating a given sensor Sᵢⱼ with an average value. Once the highest maximum value has been determined, such means determine with which sensor it is associated. The device comprises means capable of determining the morphological position of a sensor and means capable of determining from such a position a position modification instruction capable of having the maximum value disappear.

The position modification instruction is an accurate displacement instruction such as leftwards, rightwards, towards the front, towards the back, towards the front left or right or again towards the back left or right. The position modification instructions enable returning to or taking a substantially ideal position in which overpressures are suppressed.

The position modification instruction is transmitted to the patient via an oral device called tongue display unit (TDU).

In the device according to the present invention, the tongue display unit is a device attached to the patient's palate and comprising a platform with 6×6 electrodes only. Each of the patient's displacement possibilities is associated with the stimulation of a predefined group of electrodes.

For example, FIG. 3 schematically illustrates the square matrix of 6×6 electrodes. To indicate to the patient that he or she must move leftwards, six electrodes are stimulated, that is, the four central electrodes of the left-hand column and the two central electrodes of the next column. The six stimulated electrodes are hatched in FIG. 3.

In FIG. 3, the low portion is the portion attached in the palatal cavity against the upper teeth and the high portion is the portion of the tongue display unit protruding forward towards the inside of the cavity. A forward displacement will thus be preferably indicated by an activation of the electrodes of the low portion of FIG. 3 and a backward displacement will be indicated by an activation of the electrodes of the high portion of FIG. 3.

Thus, to indicate a backward and rightward displacement, the three electrodes forming an angle in the low right-hand portion of the tongue display unit when it is attached to the seated person's palate are activated. As schematically illustrated in FIG. 4, the activated electrodes then are the three hatched electrodes forming an angle between the first line and the last column of the electrode matrix.

Based en the displacement instruction thus received, the seated person may displace correspondingly.

The device according to the present invention comprises control means for checking that the maximum value having caused the instruction has disappeared. The control means comprise comparison means capable of comparing the measurement associated with the sensor having caused the instruction at the lowest threshold value of 13.332.10³ Pa (100 mm Hg). The control means measure the time elapsed from the disappearing of a maximum value. As soon as the maximum has disappeared for a determined reset time ranging between one and two minutes, the control means cause the interruption of the transmission of the displacement instruction. It should be noted that if during this control, the provided measurement exceeds again the threshold value, the measurement of the reset time period is interrupted and the instruction keeps on being provided to the seated person.

In the case where several maximum values have been detected, it should be understood by those skilled in the art that these maximum values are very likely to be morphologically close, located in a same critical area. A displacement resulting in the disappearing of the highest maximum value can accordingly result in the disappearing of several maximum values. The control means thus monitor in parallel all the previously-determined maximum values and measure if need be several reset time periods.

After the reset time period for the maximum for which a displacement instruction was provided to the seated person has elapsed, the control means determine whether the next lowest maximum is still detected. A maximum is considered as still detected if the measurement provided by the corresponding sensor is still greater than the lowest threshold value or has been lower than this threshold value for less than the reset time period.

If a maximum is still detected, then the processing means provides the seated person, via the tongue display unit, with the required displacement instruction.

For a paraplegic person, the displacement instructions can often be directly taken into account and the person him- or herself displaces as appropriate.

According to an embodiment, the present invention also comprises actuators capable of being actuated by the seated or lying person which enable him or her to modify his or her position.

It has been previously indicated that the memorization means start memorizing all the sensor measurements as soon as the measurement of a sensor has exceeded a threshold, then that control means intervene on transmission of displacement instructions. However, preferably, the control means are activated as soon as a memorization starts for a given sensor and interrupt the memorization for this sensor if the value that it provides becomes and remains lower than the lowest threshold value for the reset time period. Indeed, the reset time period necessary to guarantee that an eschar is not likely to form at the location monitored by the sensor is extremely short as compared with the time period for which an overpressure must be maintained for an eschar to be likely to form. It is not necessary to continue the memorization while the eschar-forming risk has already disappeared, which would anyway be revealed by the absence of a maximum for the considered sensor at the end of the memorization time period.

Such a device has many advantages.

First, it may easily be placed and left in place in a patient's wheelchair or bed since it is of low bulk. Indeed, the number of sensors is decreased from 900 to 144, that is, by more than a factor 6. The associated connectics is thus relatively reduced and of low bulk. Further, the device according to the present invention uses no visual display. This considerably decreases the bulk with respect to the known clinical test device.

Further, the tongue display unit exhibits decreased dimensions. Indeed, TDU devices currently used in other applications exhibit dimensions on the order of 4×4 cm capable of receiving 12×12 electrodes. The device according to the present invention uses a matrix of 6×6 electrodes of a substantially two-centimeter side.

1. A device for preventing the forming of eschars at the level of a seated or lying person's bearing area, comprising:
pressure sensors under the bearing area;
means for comparing each measurement provided by any one of the sensors with at least one threshold value;
memorization means capable of memorizing all the measurements provided by all the sensors for a first time period from an exceeding of the threshold value by at least one measurement;
means for processing the memorized measurements capable of determining, once the first time period has elapsed, displacement instructions for the seated or lying person to avoid the forming of eschars;
a tongue display unit capable of transmitting the displacement instruction to the seated or lying person; and
actuators operable by the seated or lying person and capable of causing his or her displacement.

2. The device of claim 1, further comprising:
means for determining after an exceeding of the threshold value for a given sensor whether the measurements subsequently provided by said given sensor became lower than the threshold value;
means for measuring the time for which the measurements provided by said sensor remain lower than the threshold value; and
means for interrupting for the given sensor any processing of memorization of the measurements and/or of transmission of instructions for this sensor if and as soon as the time reaches a second predetermined time period.

3. The device of claim 1 or 2, in which the value of the first time period depends on the distance from the threshold value of the measurement having exceeded said threshold value.

4. The device of claim 3, in which the threshold value is equal to 13.332.10³ Pa (100 mm Hg) and in which the first time period is equal to thirty minutes if the measured value is lower than 15.332.10³ Pa (115 mm Hg), the first time period is equal to fifteen minutes if the measured value ranges between 15.332.10³ Pa (115 mm Hg) and 17.331.10³ Pa (130 mm Hg), and in which the first time period is equal to ten minutes if the measured value is greater than 17.331.10³ Pa (130 mm Hg).

5. The device of any of claims 1 to 4, in which the pressure sensors are distributed under the bearing area at the intersection of the lines (L1, L2, L3, L4, L5, L6, L7, L8, L9, L10, L11, L12) and columns (C1, C2, C3, C4, C5, C6) of a matrix with twelve lines and twelve columns divided into two sub-matrixes with twelve lines and six columns, the interval between two successive lines of each sub-matrix being, from the rear to the front of the bearing area, 2 cm, 1.5 cm, 1.5 cm, 1 cm, 1 cm, 1 cm, 1.5 cm, 1.5 cm, 2 cm, 3 cm, and 4 cm, and the interval between two successive columns of each sub-matrix being 3.5 cm, 3 cm, 2 cm, 3 cm, and 3.5 cm.

6. The device of claim 5, in which the two sub-matrixes are adjacent.

7. The device of claim 5, in which the two sub-matrixes are separate.

8. The device of claim 5, in which the interval between the two sub-matrixes is settable on the bearing area according to the seated or lying person's morphology.

9. The device of any of claims 1 to 8, in which the tongue display unit is formed of a square matrix of thirty-six electrodes.

10. The device of any of claims 1 to 9, in which the means for processing the measurements are capable of calculating averages of the measurements memorized for the first time period, of determining and of ordering among the obtained averages the maximum values exceeding the threshold value and of determining instructions for displacing the seated or lying person to suppress said maximum values.
